(19) United States
(12) Patent Application Publication
(10) Pub. No.: US 2018/0243079 A1
(43) Pub. Date: Aug. 30, 2018

(54) POST-LUMPECTOMY BREAST IMPLANT

(71) Applicants: Jeannette POWER-COOPER, Renton, WA (US); Mark TSENG, Medina, WA (US)

(72) Inventors: Jeannette POWER-COOPER, Renton, WA (US); Mark TSENG, Medina, WA (US)

(21) Appl. No.: 15/965,504

(22) Filed: Apr. 27, 2018

Related U.S. Application Data
(63) Continuation of application No. PCT/US2016/058931, filed on Oct. 26, 2016.
(60) Provisional application No. 62/248,906, filed on Oct. 30, 2015.

(51) Int. Cl. A61F 2/12

(52) U.S. Cl. CPC A61F 2/12; A61F 2230/0071; A61F 2250/0003

(57) ABSTRACT

A post-lumpectomy breast implant can include a spherical outer shell comprising a medical-grade silicone and an inner chamber within the outer shell filled with a medical-grade silicone gel, the spherical outer shell and the inner chamber having a total volume of less than 100 cc.

### BACKGROUND

### Technical Field

The present disclosure relates generally to breast implants, and more specifically to breast implants for post-lumpectomy implantation.

### Description of the Related Art

Breast implants are often used to reconstruct a patient's breast or breasts after surgery. For example, a diagnosed breast cancer patient can have breast tissue including cancerous tissue removed, or a patient identified as being at a high-risk for developing breast cancer can have breast tissue removed. In either case, the patient may desire reconstructive surgery. A variety of breast implants are available to such patients. A variety of breast implants are also available to patients seeking cosmetic breast augmentation.

### BRIEF SUMMARY

A post-lumpectomy breast implant may be summarized as including a spherical outer shell comprising a medical-grade silicone and an inner chamber within the outer shell filled with a medical-grade silicone gel, the spherical outer shell and the inner chamber having a total volume of less than 100 cc.

### BRIEF DESCRIPTION OF THE SEVERAL VIEWS OF THE DRAWINGS

FIG. 1 illustrates a cross-sectional view of a post-lumpectomy breast implant, according to one or more illustrated embodiments.

FIG. 2 illustrates a kit of post-lumpectomy breast implants, according to one or more illustrated embodiments.

### DETAILED DESCRIPTION

Various types of breast implants are currently commercially available. For example, saline breast implants include an outer silicone shell filled with sterile saline. Saline breast implants can be relatively safe because if they leak, the saline can be easily absorbed and expelled by the human body. Silicone breast implants include an outer silicone shell filled with a silicone gel. Silicone breast implants are often considered to provide a more natural feel than saline breast implants. Form-stable breast implants, sometimes referred to as “gummy bear breast implants,” include an outer silicone shell filled with a thicker silicone gel than other silicone breast implants. Form-stable breast implants can be firmer than other breast implants, and can allow breast implants to be manufactured in a greater variety of specifically contoured, stable shapes. Form-stable breast implants can be less likely to break than other breast implants, and can maintain their shape even if the outer silicone shell breaks.

Surgical procedures available to breast cancer patients, patients at a high risk for developing breast cancer, or other patients include mastectomy, wherein the whole breast is removed, quadrantectomy, wherein a quarter of the breast is removed, and lumpectomy, wherein a small part of the breast is removed. As used herein, a “lumpectomy” can include the removal of any small portion of a breast, and includes a biopsy. Reconstructive surgery is often performed for patients who have undergone a mastectomy, but generally is not performed for patients who have undergone a lumpectomy. Patients who have undergone a lumpectomy often choose to live with the relatively small resulting deformations (relative to deformations resulting, e.g., from a mastectomy), rather than undergo further surgical procedure(s).

Thus, breast implants designed for reconstructive applications are typically shaped and sized to replace an entire human breast or a large portion of a human breast, rather than to replace a relatively small portion of a breast removed during a lumpectomy. Similarly, breast implants designed for cosmetic surgical applications are typically shaped and sized to uniformly increase the size of a natural human breast, rather than to occupy a relatively localized portion of a human breast. Thus, typical breast implants now commercially available are not suitable for reconstructive use with lumpectomy patients.

Lack of options for suitable commercially available implants may deter lumpectomy patients from undergoing reconstructive surgery after their lumpectomy, and may deter physicians from recommending the same.

Resulting lack of patient interest may in turn deter breast implant manufacturers from developing, producing, and marketing such implants. Whatever the reasons, the medical reality is that lumpectomy patients typically do not undergo reconstructive surgery after having their lumpectomy. Thus, this disclosure relates to post-lumpectomy breast implants for patients who have undergone a lumpectomy and desire reconstructive surgery.

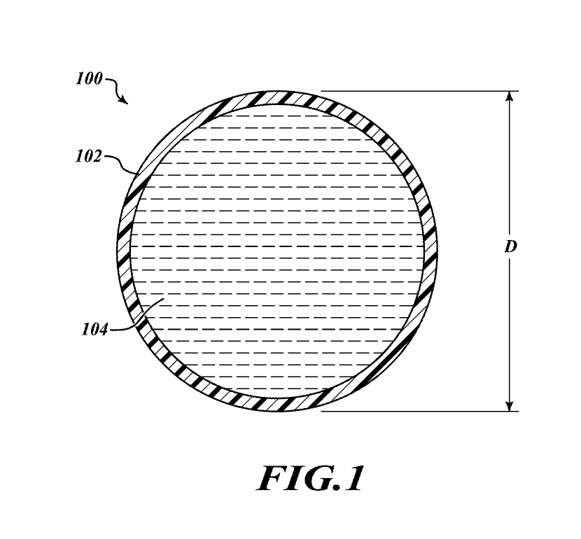

FIG. 1 illustrates a cross-sectional view of one embodiment of a post-lumpectomy breast implant 100. Breast implant 100 includes an outer sac or shell 102 made of a medical grade silicone elastomer and an interior open space or chamber 104 filled with a medical grade clear silicone gel. The breast implant 100 can be pre-filled, that is, the chamber 104 can be filled by a manufacturer prior to the implant being shipped to a physician. In some cases, the breast implant 100 can comprise only, or consist of, the outer shell 102 and the inner chamber 104 filled with the silicone gel. The breast implant 100 can be a single-walled implant 100, and can be elastically incompressible. In other embodiments, the breast implant 100 can include any of the suitable materials and features described herein. The breast implant 100 is spherical, i.e., the outer shell 102 is spherical and the inner chamber 104 is spherical. The breast implant 100 is also relatively small compared to many commercially available breast implants. For example, the breast implant 100 can have a volume less than 100 cubic centimeters (“cc” or “ml”) or a diameter D less than about 5.75 cm. The breast implant 100 can be used in reconstructive surgeries for post-lumpectomy patients.

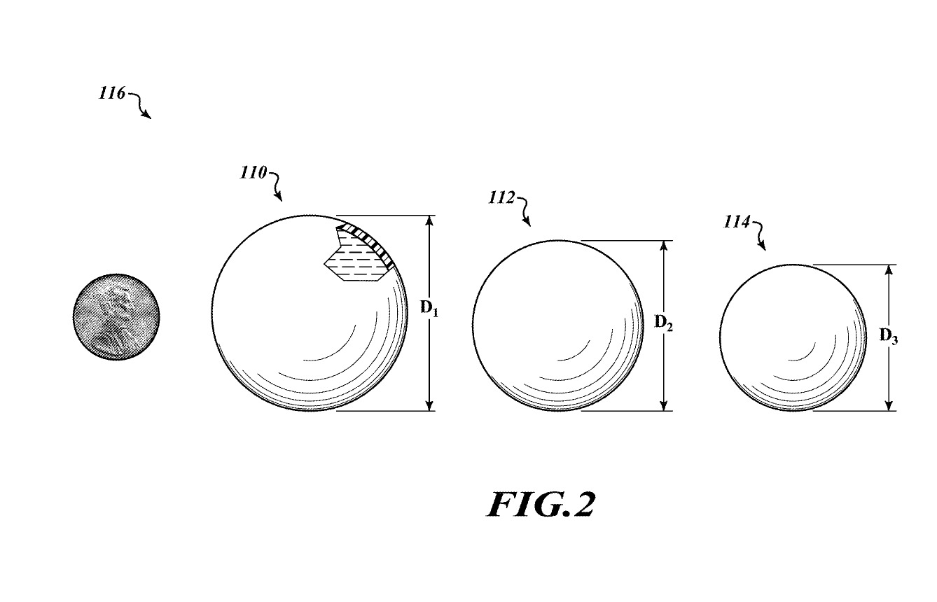

FIG. 2 illustrates three different post-lumpectomy breast implants 110, 112, and 114. Breast implants 110, 112, and 114 are the same as breast implant 100 except for their volumes, and breast implant 110 is illustrated in a partial cut-away view to show that breast implant 110 can have a structure matching that of breast implant 100. FIG. 2 illustrates that the breast implants described herein can have a variety of different volumes, such as volumes ranging from 25 cc to 95 cc. For example, the breast implants described herein can have a volume less than 100 cc, 95 cc, 90 cc, 85 cc, 80 cc, 75 cc, 70 cc, 65 cc, 60 cc, 55 cc, 50 cc, 45 cc, 40 cc, 35 cc, or 30 cc, and/or greater than 20 cc, 25 cc, 30 cc, 35 cc, 40 cc, 45 cc, 50 cc, 55 cc, 60 cc, 65 cc, 70 cc, 75 cc, 80 cc, 85 cc, or 90 cc. The breast implants described herein can be generally spherical and have a diameter D less than 5.75 cm, 5.50 cm, 5.25 cm, 5.00 cm, 4.75 cm, 4.50 cm, 4.25 cm, 4.00 cm, or 3.75 cm, and/or greater than 3.50 cm, 3.75 cm, 4.00 cm, 4.25 cm, 4.50 cm, 4.75 cm, 5.00 cm, 5.25 cm, or 5.50 cm. FIG. 2 also illustrates a penny for a possible example of scale.

FIG. 2 also illustrates a kit 116 comprising multiple breast implants 110, 112, and 114 having different volumes and hence different diameters D₁, D₂, D₃. The kit 116 can include any suitable number of breast implants, which can have any suitable volumes. For example, the kit 116 can include a plurality of breast implants each having a different volume, the volume of each breast implant in the kit differing from the volume of the others by at least 5 cc, or at least 10 cc, or at least 15 cc. In some embodiments, the kit 116 may include a plurality of post-lumpectomy breast implants having different volumes within the range of 25 cc to 95 cc, or having different diameters within a range of 3.50 cm to 5.75 cm.

A method of treating a patient can include a physician meeting and consulting with the patient and determining that the patient is a suitable candidate for a lumpectomy. The method can also include performing the lumpectomy, thereby creating a cavity or pocket in one of the patient's breasts into which a post-lumpectomy breast implant can later be implanted. The method can also include a physician (either the same physician who performed the lumpectomy or a different physician) obtaining a kit of potentially suitable (“candidate”) pre-filled post-lumpectomy breast implants, each having a different volume, and respective breast implant sizers. A breast implant sizer can include a device having a shape and volume matching that of a respective candidate post-lumpectomy breast implant, and can be temporarily inserted intraoperatively into the pocket in the patient's breast to evaluate the effect of the respective candidate breast implant on the patient's breast.

The method can also include the physician using one or more breast implant sizers of the kit to determine an appropriate size of a post-lumpectomy breast implant for use in reconstructive surgery of the patient's breast. The method can also include the physician making an initial estimate of a volume of the pocket or of a volume of a suitable post-lumpectomy breast implant, and selecting a corresponding breast implant sizer for evaluation. The method can also include the physician using the selected breast implant sizer to evaluate the effect of a respective candidate breast implant on the patient's breast. If the physician determines that the candidate breast implant is suitable for use, then the physician can proceed to implant the suitable candidate breast implant in the pocket in the patient's breast and complete the procedure. If the physician determines that the candidate breast implant is not suitable, e.g., that a larger or a smaller breast implant would be more suitable, then the physician can select another breast implant sizer accordingly and return to using the selected breast implant sizer to evaluate the suitability of a respective candidate breast implant. The physician can repeat this process until either a suitable post-lumpectomy breast implant is identified or the physician concludes that the patient is not suitable for reconstructive surgery with a post-lumpectomy breast implant.

Once a suitable post-lumpectomy breast implant is identified by the physician, the physician can implant the identified breast implant within the pocket in the patient's breast, and the physician can complete the operation. The method can include performing a lumpectomy and reconstructing the patient's breast using a post-lumpectomy breast implant in a single procedure, which can be performed in a single day. The post-lumpectomy breast implants described herein can be fabricated from silicone materials and therefore can be not bio-degradable and can prevent or resist tissue ingrowth and vascularization into the breast implant. The post-lumpectomy breast implants described herein can therefore be considered permanent and can be easily removed at a later date if removal is medically called for or otherwise desired by the patient. The post-lumpectomy breast implants described herein can also allow a physician to completely and snugly fill the pocket in the patient's breast, thereby providing pressure sufficient to prevent or reduce seroma or hematoma formation therein. The post-lumpectomy breast implants described herein can also be radiopaque, which can facilitate a physician's distinguishing between native tissue and the breast implant under x-ray.

In other embodiments, the post-lumpectomy breast implants described herein can also be used as testicular implants.

U.S. patent application Ser. No. 62/248,906, filed Oct. 30, 2015, is hereby incorporated herein by reference in its entirety.

Aspects and features of the various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A post-lumpectomy breast implant, comprising:
a spherical outer shell comprising a medical-grade silicone; and
an inner chamber within the outer shell filled with a medical-grade silicone gel;
the spherical outer shell and the inner chamber having a total volume of less than 100 cc.

2. (canceled)

3. A post-lumpectomy breast implant, consisting of:
a spherical outer shell comprising a medical-grade silicone; and
an inner chamber within the outer shell filled with a medical-grade silicone gel;
the spherical outer shell and the inner chamber having a total volume of less than 100 cc.

4. A method comprising, in a single medical procedure:
after performing a lumpectomy on a breast of a patient, reconstructing the breast of the patient using a post-lumpectomy breast implant including:
a spherical outer shell comprising a medical-grade silicone; and
an inner chamber within the outer shell filled with a medical-grade silicone gel;
the spherical outer shell and the inner chamber having a total volume of less than 100 cc.

5. The method of claim 4 wherein the lumpectomy is a biopsy.

6. The method of claim 4 wherein the inner chamber is pre-filled with the medical-grade silicone gel prior to the performing of the lumpectomy.

7. The method of claim 4, further comprising:
obtaining a kit including a first candidate post-lumpectomy breast implant having a first volume, a second candidate post-lumpectomy breast implant having a second volume larger than the first volume, a third candidate post-lumpectomy breast implant having a third volume larger than the second volume, a first breast implant sizer having a fourth volume corresponding to the first volume, a second breast implant sizer having a fifth volume corresponding to the second volume, and a third breast implant sizer having a sixth volume corresponding to the third volume.

8. The method of claim 7, further comprising:
temporarily inserting the second breast implant sizer into a pocket formed in the breast of the patient by the performing the lumpectomy.

9. The method of claim 8, further comprising:
determining that the second candidate post-lumpectomy breast implant is suitable for use in the reconstructing the breast of the patient based on the fit of the second breast implant sizer in the pocket;
removing the second breast implant sizer from the pocket; and
implanting the second candidate breast implant in the pocket.

10. The method of claim 8, further comprising:
determining that the second candidate post-lumpectomy breast implant is too small for use in the reconstructing the breast of the patient based on the fit of the second breast implant sizer in the pocket;
removing the second breast implant sizer from the pocket; and
temporarily inserting the third breast implant sizer into the pocket.

11. The method of claim 8, further comprising:
determining that the second candidate post-lumpectomy breast implant is too large for use in the reconstructing the breast of the patient based on the fit of the second breast implant sizer in the pocket;
removing the second breast implant sizer from the pocket; and
temporarily inserting the first breast implant sizer into the pocket.

12. The post-lumpectomy breast implant of claim 1 wherein the post-lumpectomy breast implant is a single-walled implant.

13. The post-lumpectomy breast implant of claim 1 wherein the post-lumpectomy breast implant is elastically incompressible.

14. The post-lumpectomy breast implant of claim 3 wherein the spherical outer shell and the inner chamber have a total volume of less than 60 cc.

15. The post-lumpectomy breast implant of claim 3 wherein the spherical outer shell and the inner chamber have a total volume of less than 30 cc.

16. The post-lumpectomy breast implant of claim 3 wherein the spherical outer shell and the inner chamber have a total volume of greater than 20 cc.

17. The post-lumpectomy breast implant of claim 3 wherein the spherical outer shell and the inner chamber have a total volume of greater than 60 cc.

18. A kit of post-lumpectomy breast implants, comprising:
a first post-lumpectomy breast implant including:
a first spherical outer shell comprising a medical-grade silicone; and
a first inner chamber within the outer shell filled with a medical-grade silicone gel;
the first spherical outer shell and the first inner chamber having a first total volume of less than 100 cc;
a second post-lumpectomy breast implant including:
a second spherical outer shell comprising a medical-grade silicone; and
a second inner chamber within the outer shell filled with a medical-grade silicone gel;
the second spherical outer shell and the second inner chamber having a second total volume of less than 100 cc; and
a third post-lumpectomy breast implant including:
a third spherical outer shell comprising a medical-grade silicone; and
a third inner chamber within the outer shell filled with a medical-grade silicone gel;
the third spherical outer shell and the third inner chamber having a third total volume of less than 100 cc.

19. The kit of post-lumpectomy breast implants of claim 18 wherein the first, second, and third total volumes differ from one another by at least 5 cc.

20. The kit of post-lumpectomy breast implants of claim 18 wherein the first, second, and third total volumes differ from one another by at least 10 cc.

21. The kit of post-lumpectomy breast implants of claim 18 wherein the first, second, and third total volumes differ from one another by at least 15 cc.
